(19) United States
(12) Patent Application Publication
(10) Pub. No.: US 2011/0165239 A1
(43) Pub. Date: Jul. 7, 2011

(54) PHARMACEUTICAL COMPOSITIONS OF ATORVASTATIN

(76) Inventors: Laman Alani, Hillsborough, CA (US); Soumojeet Ghosh, Gurnee, IL (US)

(21) Appl. No.: 13/062,891

(22) PCT Filed: Sep. 21, 2009
(86) PCT No.: PCT/US09/57647

§ 371 Date: Mar. 8, 2011

Related U.S. Application Data
(60) Provisional application No. 61/194,037, filed on Sep. 24, 2008.

(51) Int. Cl. A61K 9/24; A61K 31/40; A61K 9/20; A61P 9/10; A61K 31/66

(52) U.S. Cl. 424/472; 514/423; 424/464; 514/103

(57) ABSTRACT

The present invention provides stable pharmaceutical compositions comprised of atorvastatin and sodium bicarbonate or L-arginine. The compositions are prepared as bulk drug compositions and also as oral dosage units, such as tablets or capsules. The compositions are useful for preparation of monolithic and bi-layer tablets containing atorvastatin as the only active agent or combined with one or more additional active agents. The compositions are useful for treating hypercholesterolemia and related conditions.

### BACKGROUND OF THE INVENTION

Hypercholesterolemia and hyperlipidemia, conditions of excessively high levels of blood cholesterol and lipids, are well recognized risk factors in the onset of atherosclerosis and coronary heart disease. The blood cholesterol pool is generally dependent on dietary uptake of cholesterol from the intestine and biosynthesis of cholesterol throughout the body, especially the liver. Cholesterol is an indispensable component of virtually all cell membrane systems, as well as a precursor of a variety of steroid hormones and bile acids.

Atorvastatin calcium, which is an inhibitor of the enzyme 3-hydroxy-3-methyl glutaryl coenzyme A reductase (HMG-CoA reductase), is commercially available for the treatment of primary hypercholesterolemia, dysbetalipoproteinemia, and homozygous familial hypercholesterolemia in the U.S. and elsewhere under the trademark name LIPITOR®, (see, for example, U.S. Pat. Nos. 4,681,893; 5,273,995; 5,686,104; 5,969,156; 6,126,971, the contents of which are incorporated herein by reference in their entirety.) The active agent in LIPITOR® is atorvastatin present in a salt form, described in the Physician's Desk Reference as having the chemical name R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate, and having the following structural formula:

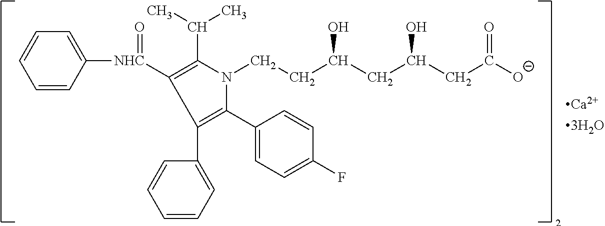

Lipitor contains a specific crystalline form of atorvastatin calcium referred to as Form I in U.S. Pat. No. 5,969,156. Atorvastatin calcium is also known to exist in amorphous and multiple polymorphic crystalline forms (see for example, U.S. Pat. Nos. 5,969,156; 6,121,461; 6,605,729; and PCT Publications WO2006/011041, WO2004/050618, WO2003/070702, WO2002/041834, WO2001/036384; the contents of each which are herein incorporated by reference in their entirety.)

However, atorvastatin is susceptible to heat, moisture, low pH environment, and light. In an acidic environment, in particular, the hydroxy acid moiety of atorvastatin will degrade to lactone. In addition, the hydroxy acid will decompose rapidly when exposed to UV or fluorescent light.

When packaged in the form of tablets, powders, granules, or within capsules, atorvastatin may be further destabilized by contact with the molecular moieties of other components. Because pharmaceutical dosage components such as binders, diluents, anti-adherents, surfactants and the like may adversely interact with atorvastatin, a need exists for improved stabilizing means for effective pharmaceutical dosages.

### SUMMARY OF THE INVENTION

The present invention provides pharmaceutical compositions comprised of atorvastatin free acid or a pharmaceutically acceptable salt thereof and an alkalizing additive selected from sodium bicarbonate and L-arginine. The compositions are prepared as bulk drug compositions and also as oral dosage units, such as tablets or capsules, and particularly tablets. The compositions are useful for preparation of monolithic tablets containing the atorvastatin as the only active agent present or combined with one or more additional active agents other than atorvastatin. The compositions are also useful for preparation of multi-layer tablets, including bi-layer tablets, wherein at least one layer contains atorvastatin as the only active agent present in the layer or combined with one or more additional active agents in the layer, and wherein each of the remaining one or more layers contain one or more active agents other than atorvastatin. The compositions are useful for the preparation of medicaments comprised of atorvastatin, and for methods of treating hypercholesterolemia and related conditions.

### DETAILED DESCRIPTION OF THE INVENTION

As used herein with respect to the compositions of this invention, the term “atorvastatin” encompasses R(R*,R*)]-2-(4-fluorophenyl)-β,δ-dihydroxy-5-(1-methylethyl)-3-phenyl-4[(phenylamino)carbonyl]-1H-pyrrole-1-heptanoic acid and pharmaceutically acceptable salts thereof, and encompasses all physical forms of the aforementioned forms. Physical form refers to the spatial order or packing of molecules, and includes for example solid crystalline, liquid-crystalline, non-crystalline and amorphous physical forms of atorvastatin and mixtures thereof. Any anhydrate or hydrate forms of atorvastatin are also encompassed. The term “atorvastatin” is not intended to be limited to the free acid, a particular pharmaceutically acceptable salt, or any particular physical form (e.g., crystalline or amorphous) unless specified otherwise, with the further exception that reference to a molar ratio of alkalizing additive to atorvastatin is meant to be calculated as a molar amount of atorvastatin on an anhydrous free acid basis, regardless of whether free acid or a salt or a hydrate/solvate form of atorvastatin (e.g., atorvastatin calcium salt, atorvastatin calcium salt trihydrate) is present in the composition. Aside from determining the molar amount of atorvastatin in a molar ratio, when the term “atorvastatin” is combined with an express description of form, then that particular form of atorvastatin is intended (for example, “atorvastatin free acid,” “atorvastatin calcium”, “amorphous atorvastatin” or “amorphous atorvastatin calcium”). Atorvastatin free acid is shown below as structural formula I:

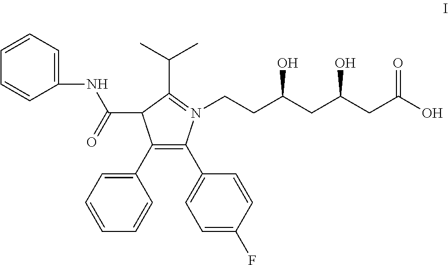

In addition to the crystalline polymorphs of atorvastatin calcium discussed supra, various salts of atorvastatin have been described, for example, in U.S. Pat. Nos. 4,681,893 and 5,273,995; and in WO2005/105738, WO2005/115980, WO2006/117761, WO2007/132472 and WO2007/063551; and crystalline forms of atorvastatin free acid are described in US2007-0276027; the contents of each which are herein incorporated by reference in their entirety. As used herein, “alkalizing additive” means an additive selected from sodium bicarbonate and L-arginine. The term “active agent” as used herein means an agent having pharmaceutical activity, as distinguished from an excipient or drug carrier.

The present invention provides pharmaceutical compositions that comprise atorvastatin and an alkalizing additive selected from L-arginine and sodium bicarbonate, wherein the molar ratio of alkalizing additive to atorvastatin is from 1:1 to 6:1. More particularly, the molar ratio of the alkalizing additive to atorvastatin is present from 1:1 to 4:1, 1:1 to 3:1, 1:1 to 2:1 or it is 1:1. More preferably, the molar ratio of the alkalizing additive to atorvastatin is 1:1. Although the two alkalizing agents can be combined in the composition as described below, in all embodiments of this invention the alkalizing agent is preferably either sodium bicarbonate or L-arginine, and more preferably it is sodium bicarbonate.

Also provided are pharmaceutical compositions that include atorvastatin, and a combination of the alkalizing additives sodium bicarbonate and L-arginine. When the combination of L-arginine plus sodium bicarbonate is used, the alkalizing additive combination is present in the same molar ratio ranges as described above, i.e., from 1:1 to 4:1, preferably from 1:1 to 3:1, and more preferably 1:1, of the alkalizing additive combination to atorvastatin. When the two alkalizing additives are used in combination in the composition, the total molar amount present of both L-arginine and sodium bicarbonate is used in calculating the alkalizing additive-to-atorvastatin molar ratio.

As used herein, molar ratio calculations are based on atorvastatin free acid equivalents [molecular weight (MW)=558.64 g/mole] present in the bulk drug used to prepare the granulation or oral dosage form composition regardless of the actual form of atorvastatin that is employed in the composition (e.g., salt, hydrate), and taking into account the amount of impurity, if any, present in the bulk drug.

Non-crystalline and crystalline forms of atorvastatin free acid are described, for example, in US Patent Application Publication US2007/0276027. However, pharmaceutically acceptable salts of atorvastatin are preferred for use in all embodiments of this invention. Suitable pharmaceutically acceptable salts of atorvastatin include but are not limited to pharmaceutically acceptable metal salts, particularly alkali metal salts such as lithium, sodium or potassium salts, and alkaline earth metal salts such as magnesium or calcium salts. Examples of pharmaceutically acceptable salts of atorvastatin and various solvate, hydrate and physical forms thereof are described in many publications, including but not limited to, WO2005/115980, WO2005/105738 and U.S. Pat. Nos. 4,681,893 and 5,273,995, each of which are herein incorporated by reference in their entirety. Atorvastatin calcium salt is the most preferred salt form. Additionally, in all embodiments of the invention atorvastatin is preferably present in the compositions in a pharmaceutically acceptable salt form as described above, wherein the salt is in either a crystalline or amorphous physical form. Crystalline or amorphous atorvastatin calcium salt is more preferred, and amorphous atorvastatin calcium salt is most preferred.

The pharmaceutical compositions of the present invention optionally include one or more excipients selected from binders, disintegrants, lubricants, surfactants, diluents, anti-oxidants, and combinations thereof. The quantity of each excipient component is expressed as a weight percentage of the total bulk or tablet composition that includes said one or more excipients, atorvastatin, and the alkalizing additive. Each of the weight percentage amounts noted for each excipient can be combined with any weight percentage amount noted for one or more of the other excipients, and all such combinations are encompassed within the scope of this invention.

Exemplary binders include, but are not limited to, hydroxypropyl cellulose, starch, polyvinyl pyrrolidone, hydroxypropyl methylcellulose, and carboxymethylcellulose, and is preferably hydroxypropyl cellulose. The binder or binder combination may be present in an amount up to 6%, e.g. from 0 to 6%, particularly from 2 to 4% and more particularly 3% by weight of the total atorvastatin, alkalizing additive and excipient composition.

Exemplary disintegrants include, but are not limited to, croscarmellose sodium and starch, and is preferably croscarmellose sodium. The disintegrant or disintegrant combination may be present in an amount up to 6%, e.g. from 0.5 to 6%, by weight, particularly from 1 to 5%, more particularly from 2 to 4%, and most particularly 3% by weight of the total composition.

Exemplary lubricants include, but are not limited to, magnesium stearate, stearic acid, palmitic acid, talc, sodium stearyl fumarate, and aerosol and is preferably magnesium stearate. The lubricant or lubricant combination may be present in the composition in an amount up to 3%, e.g. 0.25 to 3%, by weight of the total composition and particularly 0.5% by weight of the total composition. In one embodiment, the total amount of lubricant desired for the composition is divided and added at two separate stages during the process of preparing the composition, first as intragranular lubrication in the granule composition to lubricate during the granulation process step, and second as extragranular lubrication in the bulk tablet composition to provide lubrication during the tablet compression process.

Exemplary surfactants include, but are not limited to, sodium lauryl sulphate, polysorbate 80, polyoxyethylene sorbitan, and polyoxyethylene-polyoxypropylene copolymer and is preferably sodium lauryl sulfate. The surfactant or surfactant combination may be present in an amount up to 10%, e.g. from 0 to 10%, by weight of the total a composition particularly, from 0.5 to 2% by weight, and more particularly 1% by weight of the total composition.

Exemplary diluents include, but are not limited to, lactose (anhydrous or mono-hydrate), mannitol, microcrystalline cellulose, calcium phosphate dibasic, corn starch, sucrose, and silicic anhydride. The diluent or a diluent combination may be present in any amount desirable by one skilled in the art to achieve a particular tablet size or dosage strength, generally up to 90% by weight of the total composition. Preferably, the diluents microcrystalline cellulose and lactose are employed in combination in the atorvastatin composition. However, diluents can vary and are selected based on their compatibility with atorvastatin and other active agents in the composition if such additional agents are present.

Exemplary anti-oxidants include, but are not limited to, butylated hydroxyanisole (BHA), butylated hydroxytoluene (BHT), alpha tocopherol (Vitamin E) and propyl gallate. One anti-oxidant or a combination of anti-oxidants may be used. Anti-oxidant may be incorporated into the composition as a discrete component or doped onto one or more other components (excipients or active agents) which are then incorporated into the composition. In one embodiment, microcrystalline cellulose is doped with butylated hydroxyanisole (BHA) and butylated hydroxytoluene (BHT). The total amount of anti-oxidant in the composition can be for example up to 0.25%, e.g. 0.05 to 0.25%, or more particularly 0.1%, by weight of the total composition. While one or more anti-oxidants can be added, it has been found that anti-oxidants are not necessary to include in the compositions of this invention in order to achieve desirable stability. Accordingly, anti-oxidants are preferably omitted from the atorvastatin compositions described herein.

In another embodiment of this invention, the composition comprises the alkalizing additive and atorvastatin in the range of molar ratios described supra, with diluent, binder, disintegrant, surfactant and lubricant. In a further embodiment the composition comprises sodium bicarbonate and amorphous atorvastatin calcium salt, in a sodium bicarbonate to atorvastatin molar ratio range from 1:1 to 4:1, lactose (anhydrous), microcrystalline cellulose, hydroxypropyl cellulose, croscarmellose sodium, sodium lauryl sulfate and magnesium stearate. In a class of this embodiment, the sodium bicarbonate to atorvastatin molar ratio range is from 1:1 to 3:1, and in a sub-class it is 1:1. Another embodiment of the present invention includes a pharmaceutical composition which comprises L-Arginine and amorphous atorvastatin calcium salt, in an L-arginine to atorvastatin molar ratio range from 1:1 to 4:1, lactose (anhydrous), microcrystalline cellulose, hydroxypropyl cellulose, croscarmellose sodium, sodium lauryl sulfate, and magnesium stearate. In a class of this embodiment, the L-arginine to atorvastatin molar ratio range is from 1:1 to 3:1, and in a sub-class it is 1:1.

As used herein, the terms “pharmaceutical composition” and “composition” encompass both the bulk granulation composition and individual oral dosage units (tablets, capsules, pills and the like) comprised of atorvastatin or atorvastatin combined with one or more additional active agents, with the pharmaceutically inactive excipients described herein (the active agent or agents and the excipients including the alkalizing additive are collectively referred to herein as the “components” of the composition). The bulk granulation composition is material that has not yet been formed into individual oral dosage units. The oral dosage unit form of the pharmaceutical composition is preferably a tablet.

The total weight of the bulk composition or oral dosage unit comprised of the components described herein will necessarily vary according to the amount of bulk granulation composition or the size of the oral dosage unit that is desired to be produced. For the purpose of calculating the weight percentage of each of the components that comprise any given amount of bulk granulation composition or an individual oral dosage unit, the weights of all the components (i.e., the atorvastatin, the optional one or more additional active agents, and the excipients) in a given amount of bulk granulation or in an oral dosage unit are added together to determine the total weight of the composition. As would be understood in the art, the final bulk granulation composition would not contain either solvents used in the granulation process nor coating materials as components. Therefore, for the purpose of calculating the total weight of the composition in either bulk granulation or oral dosage unit form in order to calculate the weight percentage of each component, coating materials and solvents are not considered components and would not be included in the total weight calculation of the composition.

It is understood in the art that component weight ranges and specific weight amounts used herein to describe the composition of a single oral dosage unit can be scaled proportionally to make bulk composition. Of course, the component weight percentage amounts used herein are applicable to either individual oral dosage units or to bulk granulation composition.

Other than in the operating examples, or where otherwise indicated, all numbers expressing quantities of ingredients, reaction conditions, and so forth used in the specification and claims are to be understood as being modified in all instances by the term “about.”

The pharmaceutical compositions of the present invention optionally include one or more active agents in a therapeutically effective amount in addition to the atorvastatin. The amount of additional active agent or agents which may be present will depend on the therapeutically desirable amount of additional active agent or agents and therapeutically desirable amount of atorvastatin per dosage unit, maximum feasible dosage unit size, and the physical and chemical properties of the optional additional active agent or agents.

Suitable additional active agents include but are not limited to those that can be co-administered with atorvastatin. Exemplary additional active agents include anti-atherosclerotic agents; anti-diabetes agents; anti-obesity agents; anti-hypertensive agents; agents used for the treatment of metabolic syndrome; lipid modifying agents; agents that have both lipid-modifying effects and other pharmaceutical activities; cholesterol absorption inhibitors such as ezetimibe (ZETIA®) which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. RE37721 and 5,846,966; HDL-raising agents such as cholesterol ester transfer protein (CETP) inhibitors, for example anacetrapib also known as MK-859 having chemical name (4S,5R)-5-[3,5-bis(trifluoromethyl)phenyl]-3-{[4′-fluoro-2′-methoxy-5′-(1-methylethyl)-4-(trifluoromethyl)[1,1′-biphenyl]-2-yl]methyl}-4-methyl-2-oxazolidinon (described in WO2006/014357, Merck & Co. Inc.) and JTT-705 (Japan Tobacco Company); reverse cholesterol transport enhancers; other HMG-CoA synthase inhibitors; diuretics, for example hydrochlorothiazide; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; niacin in immediate and controlled release form, optionally with an anti-flushing agent such as a DP antagonist such as laropiprant, and particularly the product known as TREDAPTIVE® which contains controlled-release niacin in one layer and laropiprant in the other layer of a bilayer tablet; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate and gemfibrozil; PPAR dual α/γ agonists such as muraglitazar; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan and losartan with hydrochlorothiazide; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all subtypes of this receptor, such as LXRα and LXRβ; bisphosphonate compounds such as alendronate sodium; cyclooxygenase-2 inhibitors such as rofecoxib and celecoxib; sibutramine; orlistat; topiramate; naltrexone; bupriopion; phentermine; phentermine/topiramate combination (QNEXA®); NPY5 antagonists; Acetyl-CoA Carboxylase-1 and -2 (ACC) inhibitors; MCH1R antagonists; CB1 antagonists/inverse agonists such as those described in WO03/077847 and WO05/000809; DPP-4 (dipeptidylpeptidase-4) inhibitors such as sitagliptin (JANUVIA®) and vildagliptin (GALVUS®); sulfonylureas such as chlorpropamide, tolazamide, glyburide, glipizide, and glimepiride; biguanides, such as metformin; alpha-glucosidase inhibitors such as acarbose and miglitol; meglitinides such as repaglinide; glucagon-receptor agonists; and glucokinase activators. This list of active agents is illustrative and not intended to be exhaustive.

The oral dosage unit compositions of the present invention can have any form suitable for oral administration. Preferably, the composition is in the form of a tablet or a capsule. Exemplary tablets include monolithic (i.e., single layer), bi-layer and multi-layer tablets. In one embodiment, a monolithic tablet comprises atorvastatin and sodium bicarbonate or L-arginine optionally admixed with excipients and one or more additional active agents then compressed into a tablet, for example wherein atorvastatin and sodium bicarbonate or L-arginine are admixed in a composition with a cholesterol absorption inhibitor (e.g. ezetimibe), a high density lipoprotein (HDL) raising agent (e.g., niacin in immediate or extended release form), a CETP inhibitor (e.g. anacetrapib), or an anti-diabetic agent (e.g. sitagliptin).

Alternatively, a multi-layer tablet, particularly a bilayer tablet, can be employed. In bilayer and multi-layer embodiments, each tablet layer may contain one or more pharmaceutically active agents, selected independently of the agents present in any other tablet layer. In one embodiment, an atorvastatin composition of this invention containing the alkalizing additive can comprise one layer and a different composition containing additional active agent or agents can comprise the second layer of a bi-layer tablet. For example, a composition of this invention comprising atorvastatin and sodium bicarbonate or L-arginine is present in one layer and the second layer is comprised of a cholesterol absorption inhibitor such as ezetimibe, niacin (immediate or extended release), a CETP-inhibitor such as anacetrapib, or an anti-diabetic agent such as sitagliptin. Another exemplary bi-layer tablet comprises extended-release niacin in one layer, and a composition of this invention containing atorvastatin and sodium bicarbonate or L-arginine admixed with a DP-antagonist, particularly laropiprant, in the second layer. The type of solid dosage form can be determined by one of skill in the art based upon a variety of considerations such as, for example, the compatibility or desired release profile of the active agents and the desired therapeutic effect.

The pharmaceutical compositions of the present invention optionally include a film coating. The coating may be present on the granule composition surface (wherein the coating material is added to bulk granulation prior to forming oral dosage forms) and/or the tablet surface (wherein the coating is applied to the exterior surface of a compressed tablet or core comprised of atorvastatin and optionally one or more pharmaceutically acceptable excipients). Where both granule and tablet coating are present, the compositions of both coatings may be selected independently. Exemplary coatings include those containing lactose, hydroxypropyl methylcellulose, hydroxypropyl cellulose, hydroxypropyl methylcellulose, polyvinyl alcohol, polyethylene glycol or crosscarmellose sodium or sodium carboxymethylcellulose. Preferred coatings include those containing a combination of lactose and hydroxypropyl methylcellulose; a combination of hydroxypropyl cellulose and hydroxypropyl methylcellulose; or a combination of polyvinyl alcohol and polyethylene glycol. More preferred coatings include those containing hydroxypropyl cellulose and hydroxypropyl methylcellulose; or polyvinyl alcohol and polyethylene glycol. Optionally, a colorant may be present in a coating. Coatings are preferred to mask the unpleasant taste of the atorvastatin and other optional active agents. Use of a tablet surface coating is also preferred for bilayer and multi-layer tablets to reduce the possibility of delamination. When present, a film coating preferably comprises from about 1 to 6% by weight of the final coated tablet, although higher weight amounts, e.g. up to 10% or more, can be used. For calculating the percentage weight of the coating, the final coated tablet weight includes the weight of the components plus the weight of the coating. More preferably, a film coating comprises 2 to 4% of the final coated tablet weight. Most preferably, a coating comprises about 3% of the final coated tablet weight. Coatings having low or no oxygen permeability are desirable for further reducing oxidative degradation of atorvastatin, particularly amorphous atorvastatin, and may tend toward the higher weight range to be effective, i.e, up to 10%.

The atorvastatin compositions described herein will provide immediate release of the drug after administration as that term is understood in the art, but the compositions can be formulated to modify the release rate to achieve controlled, extended or delayed release and the like (collectively referred to herein as controlled release). Controlled release dosage forms can be prepared by methods known to those skilled in the art, for example, by granule or tablet enteric coatings or by admixture of a controlled release matrix component in the composition. Where the atorvastatin and sodium bicarbonate or L-arginine are present in combination with one or more additional pharmaceutically active agents, the release rate of any pharmaceutical agent present may be controlled, independent of the release of other agents. For example, a fixed-dose combination composition containing atorvastatin and an alkalizing additive admixed with one or more additional pharmaceutically active agents may have an immediate release or controlled release tablet dissolution profile. Alternatively, in a fixed-dose combination composition containing atorvastatin and an alkalizing additive in one layer of a bi-layer or multi-layer tablet and one or more additional pharmaceutically active agents present in an additional layer(s), each individual tablet layer can be immediate release or controlled release independent of any other layer. A preferred embodiment is a bi-layer tablet with one layer that provides immediate-release which is comprised of atorvastatin and the alkalizing additive and optionally contains another active agent, and a second layer that provides immediate or controlled-release which is comprised of an active agent other than atorvastatin. In another embodiment, the atorvastatin and the alkalizing additive are present in a controlled release form in either a monolithic oral dosage form or in one layer of a bi-layer oral dosage form.

Compositions of the present invention are optionally and preferably stored in protective packaging to minimize or prevent degradation of the composition due to exposure to oxygen and/or water. When present, suitable packaging includes desiccants, oxygen scavengers, anti-oxidants, vacuum packing, nitrogen header space and the like. For example, bilayer tablets of the present invention containing amorphous atorvastatin calcium salt can be stored in a container such as a sealed bottle or foil pouch which contains an oxygen scavenger with or without a desiccant to reduce degradation during storage.

Also provided is a method for treating hypercholesterolemia (heterozygous familial and nonfamilial), mixed dyslipidemia (Fredrickson Types IIa and IIb), homozygous familial hypercholesterolemia or a combination thereof as well as treating heterozygous familial hypercholesterolemia in pediatric patients 10-17 years of age, in a patient by administering to the patient a therapeutically effective amount of a composition of the present invention. When an additional active agent is present in the composition, there is also provided a method of treatment for disorders in addition to those noted above, such as diabetes, obesity, etc., which will depend on the selection of the active agent for co-administration.

The percentage of atorvastatin, and optional additional active agent(s) in the compositions of the present invention may be varied, it being necessary that it should constitute a proportion such that a suitable dosage shall be obtained. A dose employed may be determined by a physician or qualified medical professional, and depends upon the desired therapeutic effect, the duration of the treatment, and the condition of the patient. The atorvastatin is preferably administered to the patient once a day at a dose of 5, 10, 20, 40, or 80 mg per day, on an atorvastatin free acid weight basis. In each particular case, the doses are determined in accordance with the factors distinctive to the patient to be treated, such as age, weight, general state of health and other characteristics which can influence the efficacy of the atorvastatin, and optional additional active agent(s).

The following non-limiting Examples illustrate certain aspects of the invention. The following notations used in the Tables are defined as follows:
* Weight percentage (Wt %) amounts represent the relative weight of bulk materials used in the composition. For atorvastatin, this value represents the weight of atorvastatin calcium salt material, including any impurities such as e.g. water content, incorporated into the bulk batch.
** Mole amounts represent the actual amount of pure atorvastatin on a free acid basis (MW=558.64 g/mole).
   The actual weight % of bulk atorvastatin calcium salt in the following Example compositions may vary among compositions, even though the mole amount of pure atorvastatin on a free acid basis that is present in a given quantity (e.g., 100 g) of each composition is the same. This is due to different amounts of impurity present in different batches of atorvastatin calcium bulk material. The appropriate weight % of bulk atorvastatin calcium is used in each composition to achieve the desired mole amount of pure atorvastatin on a free acid basis. One (1.000) mg atorvastatin free acid=1.036 mg atorvastatin calcium salt.

### Example 1

### Atorvastatin and Sodium Bicarbonate Compositions

A 10 kg quantity of the composition of Formulation A (shown in Table 1) was prepared by first weighing out 1450 g of amorphous atorvastatin (calcium salt), 2800 g of lactose (anhydrous), 4798 g of microcrystalline cellulose, 199.9 g of sodium bicarbonate, 300.8 g of hydroxypropyl cellulose, 300.1 g of croscarmellose sodium, 100.0 g of sodium lauryl sulfate, and 50.8 g of magnesium stearate. Atorvastatin, lactose (anhydrous), microcrystalline cellulose, sodium bicarbonate, hydroxypropyl cellulose, croscarmellose sodium, and sodium lauryl sulfate were blended in a suitable tumble blender (40L Bohle blender) for 10 minutes in a controlled humidity room (relative humidity ≦30%). Magnesium stearate was then added to the batch, and blended for an additional 5 minutes to create a lubricated blend. The lubricated blend was transferred to a suitable roller compactor (Alexanderwerk WP120, 40 mm roll) and compacted at a suitable pressure (40-80 bar) and suitable roll speed (3-10 rpm), maintaining a suitable roll gap (1.5-3 mm) in a humidity controlled room (relative humidity ≦30%). Ribbons generated from the roller compaction step were then milled using a suitable mill (Alexanderwerk WP120 in-line RFG) with a suitable primary mill screen choice (Range: 2.5-1.6 mm) and a suitable secondary mill screen choice (Range: 1-0.4 mm). The resulting granules were then stored in a double polyethylene bag with 50 lg silicone desiccant canisters, at −20° C.

Formulation β is prepared according to the procedure described above, with quantities of materials adjusted according to the composition described in Table 1 below to achieve a 10 kg batch scale. Formulation C (Table 1) was prepared according to the procedure described above at a 12 kg scale, with 324 g of amorphous atorvastatin (calcium salt), 8931 g of lactose (anhydrous), 1800 g of microcrystalline cellulose, 44.4 g of sodium bicarbonate, 360 g of hydroxypropyl cellulose, 360 g of croscarmellose sodium, 120 g of sodium lauryl sulfate, and 60 g of magnesium stearate.

TABLE 1
Atorvastatin/Sodium bicarbonate compositions

| | Formulation | | | | | |
|---|---|---|---|---|---|---|
| | A | | B | | C | |
| | Weight % | Moles | | Moles | | Moles |
| | (Wt %)* | (per 100 g) | Wt %* | (per 100 g) | Wt %* | (per 100 g) |
| Atorvastatin calcium | 14.51 | 0.02** | 14.51 | 0.02** | 2.72 | 0.004** |
| (amorphous) | | | | | | |
| Lactose, anhydrous | 28 | | 65.00 | | 74.43 | |
| Microcrystalline cellulose | 48 | | 11.05 | | 15 | |
| Hydroxypropyl cellulose | 3 | | 3 | | 3 | |
| Croscarmellose sodium | 3 | | 3 | | 3 | |
| Sodium lauryl sulfate | 1 | | 1 | | 1 | |
| Sodium bicarbonate | 2 | 0.02 | 2 | 0.02 | 0.37 | 0.004 |
| Magnesium Stearate | 0.5 | | 0.5 | | 0.5 | |
| Total: | 100% | | 100% | | 100% | |
| Batch Scale: | 10 kg | | 10 kg | | 12 kg | |

1.000 mg atorvastatin free acid = 1.036 mg atorvastatin calcium salt

### Example 2

### Atorvastatin and L-Arginine Compositions

Step A: A 7.5 kg quantity of the composition of Formulation D (shown in Table 2) is prepared by first weighing out 1088.25 of amorphous atorvastatin (calcium salt), 2048.06 g of lactose (anhydrous), 3441.75 g of microcrystalline cellulose, 307.5 g of L-arginine, 225 g of hydroxypropyl cellulose, 225 g of croscarmellose sodium, 75 g of sodium lauryl sulfate, and 37.5 g of magnesium stearate.

Step B: Atorvastatin, lactose (anhydrous), microcrystalline cellulose, L-arginine, hydroxypropyl cellulose, croscarmellose sodium, and sodium lauryl sulfate are blended in a suitable tumble blender (40 L Bohle blender) for 10 minutes in a controlled humidity room (relative humidity ≦30%). Half of the magnesium stearate is then added to the batch and blended for an additional 5 minutes. The lubricated blend is transferred to a suitable roller compactor (Alexanderwerk WP120, 40 mm roll) and compacted at a suitable pressure (40-80 bar) and suitable roll speed (3-10 rpm), maintaining a suitable roll gap (1.5-3 mm) in a humidity controlled room (relative humidity ≦30%). Ribbons generated from the roller compaction step are then milled using a suitable mill (Alexanderwerk WP120 in-line RFG) with a suitable primary mill screen choice (Range: 2.5-1.6 mm) and a suitable secondary mill screen choice ((Range: 1-0.4 mm). The resulting granules are then stored in a double polyethylene bag with 50 lg silicone desiccant canisters, at −20° C.

After completion of the granulation step and prior to compressing into tablets, the formulation is extragranularly lubricated by adding the remaining quantity of magnesium stearate (half of the original amount) and blending for an additional 5 minutes in a suitable blender.

Formulations E and F (Table 2) are prepared according to the procedure described above.

TABLE 2
Atorvastatin/L-arginine compositions (weight % of composition)

| | Formulation | | | | | |
|---|---|---|---|---|---|---|
| | D | | E | | F | |
| | | Moles | | Moles | | Moles |
| | Wt %* | (per 100 g) | Wt %* | (per 100 g) | Wt %* | (per 100 g) |
| Atorvastatin calcium | 14.51 | 0.02** | 14.51 | 0.02** | 2.72 | 0.004** |
| (amorphous) | | | | | | |
| Lactose, anhydrous | 28 | | 67.89 | | 74.01 | |
| Microcrystalline cellulose | 45.89 | | 6 | | 15 | |
| Hydroxypropyl cellulose | 3 | | 3 | | 3 | |
| Croscarmellose sodium | 3 | | 3 | | 3 | |
| Sodium lauryl sulfate | 1 | | 1 | | 1 | |
| L-arginine | 4.1 | 0.02 | 4.1 | 0.02 | 0.768 | 0.004 |
| Magnesium Sterate | 0.5 | | 0.5 | | 0.5 | |
| Total: | 100.00% | | 100.00% | | 100.00% | |
| Batch Scale: | 7.5 kg | | 7.5 kg | | 7.5 kg | |

1.000 mg atorvastatin free acid = 1.036 mg atorvastatin calcium salt

### Example 3

### Preparation of Microcrystalline Cellulose Doped with BHA and BHT

To prepare 3 kg batch of doped microcrystalline cellulose, 450 g of purified water are mixed with 1800 g of pure ethanol, using a suitable mixer (Lightnin Mixer), at a suitable impeller speed (100 to 400 rpm). 7.5 g of butylated hydroxyanisole (BHA), and 7.5 g of butylated hydroxytoluene (BHT) are then dissolved in the ethanol-water mixture, using a suitable mixer (Lightnin Mixer). 2985 g of microcrystalline cellulose are then transferred to a suitable high shear mixer (40 L Diosna). The BHA and BHT-containing water-ethanol mixture is then sprayed onto the microcrystalline cellulose in the high shear mixer, at a suitable spray rate (1 to 100 g/min), with a suitable mixer speed (Range: 50-2001 rpm) and chopper speed (500 to 2000 rpm). The doped microcrystalline cellulose from this step is then transferred to a suitable dryer (GPCG 15) where the water and ethanol are evaporated off.

### Example 4

### Atorvastatin/Sodium Bicarbonate and Atorvastatin/L-Arginine Compositions with Doped Microcrystalline Cellulose

A 125 g quantity of the atorvastatin/sodium bicarbonate and doped microcrystalline cellulose composition (Table 3) was prepared by first dispensing 17.27 g amorphous atorvastatin (calcium salt), 25.00 g lactose (anhydrous), 63.99 g doped microcrystalline cellulose prepared according to Example 3, 3.75 g sodium bicarbonate, 3.75 g hydroxypropyl cellulose, 1.25 g croscarmellose sodium, and 2.50 g sodium lauryl sulfate. Atorvastatin, lactose (anhydrous), doped microcrystalline cellulose, sodium bicarbonate, hydroxypropyl cellulose, croscarmellose sodium, and sodium lauryl sulfate were blended in a suitable tumble blender (1 L PK or Turbula blender) for 10 minutes in a controlled humidity room (relative humidity ≦30%). Half of the magnesium stearate was then added to the batch and blended for an additional 5 minutes. The lubricated blend was transferred to a suitable roller compactor (TFC Labo, 15 mm roll) and compacted at a suitable pressure (20-80 bar), suitable roll speed (3-10 rpm), maintaining a suitable roll gap (1.5-3 mm) in a humidity controlled room (relative humidity ≦30%). Ribbons generated from the roller compaction step were then milled using a suitable mill (TFC Lab-associated RFG) with a suitable primary mill screen choice (1.6 to 2.5 mm) and a suitable secondary mill screen choice (0.4 to 1 mm). The resulting granules were then stored in a double polyethylene bag with 50 lg silicone desiccant canisters, at −20° C.

After completion of the granulation step and prior to compressing into tablets, the formulation is extragranularly lubricated by adding the remaining quantity of magnesium stearate (half of the original amount) and blending for an additional 5 minutes in a suitable blender.

The atorvastatin/L-arginine with doped microcrystalline cellulose formulation (Table 4) was prepared according to the procedure described above, substituting the quantities and materials described in Table 4 below.

TABLE 3
Atorvastatin/sodium bicarbonate with doped microcrystalline cellulose

| | | Weight | Weight | Moles |
|---|---|---|---|---|
| | Wt % | (mg/tab) | (g) | (per 100 g) |
| Atorvastatin calcium | 13.81 | 82.88 | 17.27 | 0.02** |
| (amorphous) | | | | |
| Lactose | 20.00 | 120.00 | 25.00 | |
| Microcrystalline Cellulose | 51.19 | 307.14 | 63.99 | |
| (Doped with 0.25% BHA | | | | |
| and 0.25% BHT by weight) | | | | |
| Hydroxypropyl Cellulose | 3.00 | 18.00 | 3.75 | |
| Croscarmellose Sodium | 3.00 | 18.00 | 3.75 | |
| Sodium Bicarbonate | 6.00 | 36.00 | 7.50 | 0.07 |
| Sodium Lauryl Sulphate | 1.00 | 6.00 | 1.25 | |
| Magnesium Stearate | 2.00 | 12.00 | 2.50 | |
| Butylated Hydroxy Anisole | 0.00 | 0.77 | *** | |
| (BHA) | | | | |
| Butylated Hydroxy Toluene | 0.00 | 0.77 | *** | |
| (BHT) | | | | |
| Total | 100.00 | 600.02 | 125.00 | |

*** Weights of BHA and BHT are included in the Microcrystalline Cellulose weight.

TABLE 4
Atorvastatin/L-arginine with doped microcrystalline cellulose

| | | Weight | Weight | Moles |
|---|---|---|---|---|
| | Wt % | (mg/tab) | (g) | (per 100 g) |
| Atorvastatin calcium | 13.81 | 82.88 | 17.27 | 0.02** |
| (amorphous) | | | | |
| Lactose | 20.00 | 120.00 | 25.00 | |
| Microcrystalline Cellulose | 43.37 | 260.22 | 54.21 | |
| (Doped with 0.25% BHA | | | | |
| and 0.25% BHT by weight) | | | | |
| Hydroxypropyl Celluose | 3.00 | 18.00 | 3.75 | |
| Croscarmellose Sodium | 3.00 | 18.00 | 3.75 | |
| L-Arginine | 13.81 | 82.88 | 17.27 | 0.08 |
| Sodium Lauryl Sulphate | 1.00 | 6.00 | 1.25 | |
| Magnesium Stearate | 2.00 | 12.00 | 2.50 | |
| Butylated Hydroxy Anisole | 0.00 | 0.65 | *** | |
| (BHA) | | | | |
| Butylated Hydroxy Toluene | 0.00 | 0.65 | *** | |
| (BHT) | | | | |
| Total | 100.00 | 599.98 | 125.00 | |

*** Weights of BHA and BHT are included in the Microcrystalline Cellulose weight.

### Example 5

### The Compositions Described in Tables 5 and 6 were Prepared Using Essentially the Same Processes Described in Prior Examples

TABLE 5
Amorphous Atorvastatin Calcium Salt Granulation
Composition, 13% Drug Load

| | | Mass | | Moles |
|---|---|---|---|---|
| | | (100 | MW | (100 g |
| | Wt % | g batch) | (g/mole) | batch) |
| Atorvastatin calcium, | 14.62% | 14.62 | 577.688 | 0.0253 |
| (amorphous) (bulk) [symbol] | | | | |
| Free Acid Equivalent [symbol] [symbol] | 13.33% | 13.33 | 558.64 | 0.0239 |
| Microcrystalline cellulose | 48.00% | 48.00 | 36000 | 0.0013 |
| Lactose, Anhydrous | 27.88% | 27.88 | 342.3 | 0.0814 |
| Hydroxypropyl Cellulose | 3.00% | 3.00 | 50000 | 0.0001 |
| Croscarmellose Sodium | 3.00% | 3.00 | 90000 | 0.0000 |
| Sodium Bicarbonate | 2.00% | 2.00 | 84.01 | 0.0238 |
| Sodium Lauryl Sulfate | 1.00% | 1.00 | 420 | 0.0024 |
| Magnesium Stearate | 0.50% | 0.50 | 591.27 | 0.0008 |

[symbol]

1.097 mg bulk drug = 1.036 mg atorvastatin calcium salt = 1.000 mg atorvastatin free acid [symbol] [symbol]
Free Acid Equivalent = the amount of atorvastatin free acid present in the composition based on the amount of bulk drug/atorvastatin calcium salt present in the composition

TABLE 6
Amorphous Atorvastatin Calcium Salt Granulation
Composition, 2.5% Drug Load

| | | Mass | | Moles |
|---|---|---|---|---|
| | | (100 g | MW | (100 g |
| | Wt % | batch) | (g/mole) | batch) |
| Atorvastatin calcium, | 2.74% | 2.74 | 577.688 | 0.0047 |
| (amorphous) (bulk) [symbol] | | | | |
| Free Acid Equivalent [symbol] [symbol] | 2.50% | 2.50 | 558.64 | 0.0045 |
| Microcrystalline cellulose | 15.00% | 15.00 | 36000 | 0.0004 |
| Lactose, Anhydrous | 74.39% | 74.39 | 342.3 | 0.2173 |
| Hydroxypropyl Cellulose | 3.00% | 3.00 | 50000 | 0.0001 |
| Croscarmellose Sodium | 3.00% | 3.00 | 90000 | 0.0000 |
| Sodium Bicarbonate | 0.37% | 0.37 | 84.01 | 0.0044 |
| Sodium Lauryl Sulfate | 1.00% | 1.00 | 420 | 0.0024 |
| Magnesium Stearate | 0.50% | 0.50 | 591.27 | 0.0008 |

[symbol]

1.097 mg bulk drug = 1.036 mg atorvastatin calcium salt = 1.000 mg atorvastatin free acid [symbol] [symbol]
Free Acid Equivalent = the amount of atorvastatin free acid present in the composition based on the amount of bulk drug/atorvastatin calcium salt present in the composition

### Example 6

### Pharmacokinetic Studies

Additional amorphous atorvastatin/sodium bicarbonate and amorphous atorvastatin/L-arginine compositions were prepared at 125 g scale according to the process described in Example 4 and the compositions described in Table 7. Upon completion of the granulation step, both formulations were extragranularly lubricated by adding the remaining quantity of magnesium stearate (half of the original amount) and blending for an additional 5 minutes in a suitable blender (1 L PK or Turbula blender), compressed into tablets on a suitable tablet press (Korsch press) under suitable conditions ( 8/32″ standard concave tooling, 25 RPM turret speed, 3-14 kN compaction force), and submitted for animal testing in dogs, in conjunction with the comparator (40 mg Lipitor®).

Six male Beagle dogs were divided into two groups for a two-period biocomparison study, with the first group receiving both the Lipitor® comparator and the sodium bicarbonate formulations described in Table 7 below in a two-way crossover study, and the second group receiving both the Lipitor® comparator and the L-arginine formulations described in Table 7 in a two-way cross-over study. In the second study period, the experimental formulation administered to each study group were transposed, but again administered with the Lipitor control in a two-way crossover manner. Both formulations were thus tested against the control in all 6 dogs.

The dogs were fasted overnight and treated intramuscularly with pentagastrin thirty minutes prior to administering a 40 mg dose of atorvastatin via the Lipitor® comparator, sodium bicarbonate, or L-arginine composition, as described above. Food was returned 4 hours after dosing, and blood was drawn at pre-dose, 0.25, 1, 2, 4, 6, 8, and 24 hours after dosing. The pharmacokinetic data obtained for this study is listed in Table 7.

TABLE 7
Pharmacokinetic data from dog study

| | | Sodium | | | | |
|---|---|---|---|---|---|---|
| | | Bicarbonate Formulation^{†} | | L-arginine Formulation^{†} | | |
| | | | Moles | | Moles | |
| | Lipitor ® | Wt % | per 100 g | Wt % | per 100 g | MW |
| Atorvastatin calcium | — | 13.81^{Θ} | 0.0239** | 13.81^{Θ} | 0.0239** | 558.64 |
| (amorphous) | | | | | | |
| Lactose | — | 28 | | 28 | | |
| Microcrystalline | — | 46.95 | | 44.864 | | |
| Cellulose | | | | | | |
| Hydroxypropyl | — | 3 | | 3 | | |
| Celluose | | | | | | |
| Croscarmellose | — | 3 | | 3 | | |
| Sodium | | | | | | |
| Sodium Bicarbonate | — | 2 | 0.0238 | 0 | 0 | 84.01 |
| L-arginine | — | 0 | 0 | 4.1 | 0.0235 | 174.20 |
| Sodium Lauryl | — | 1 | | 1 | | |
| Sulphate | | | | | | |
| Magnesium Stearate | — | 2 | | 2 | | |
| Butylated Hydroxy | — | 0.12 | | 0.112 | | |
| Anisole | | | | | | |
| Butylated Hydroxy | — | 0.12 | | 0.112 | | |
| Toluene | | | | | | |

| | | |
|---|---|---|
| Batch Size | 125 g | 125 g |

Animal Biostudy #1

| | | | |
|---|---|---|---|
| AUC (nM*hr) | 121.9 | 217 | — |
| C-max (nM) | 53.1 | 67.4 | — |
| T-max (hr) | 0.5 | 2 | — |
| AUC Ratio | 1 | 1.8 | |
| (relative to Lipitor ®) | | | |

Animal Biostudy #2

| | | | |
|---|---|---|---|
| AUC (nM*hr) | 157.5 | — | 139.2 |
| C-max (nM) | 61.4 | — | 36.4 |
| T-max (hr) | 0.5 | — | 2 |
| AUC Ratio | 1 | — | 0.9 |
| (relative to Lipitor ®) | | | |

^{†}1:1 molar ratio of atorvastatin free acid:alkalizing additive
^{Θ}Based on MW of atorvastatin calcium salt without impurity

Atorvastatin/sodium bicarbonate and atorvastatin/L-arginine compositions listed in Table 8 were prepared according to Example 4 at a 400 g batch size, extragranularly lubricated and compressed into tablets as described above, and submitted for a human biocomparison study with the comparator (40 mg Lipitor®). Thirty-six healthy adult subjects were administered 40 mg atorvastatin in a 3-way cross-over study, via Lipitor® comparator, sodium bicarbonate, and L-arginine compositions, described in Table 8 below. Each formulation was thus tested in each study subject. The pharmacokinetic data obtained from this study is listed in Table 8.

TABLE 8
Pharmacokinetic data from human bio comparison study

| | | Sodium | | | | |
|---|---|---|---|---|---|---|
| | | Bicarbonate Formulation^{†} | | L-arginine Formulation^{†} | | |
| | | | Moles | | Moles | |
| | Lipitor ® | Wt % | per 100 g | Wt % | per 100 g | MW |
| Atorvastatin calcium | — | 13.81^{Θ} | 0.0239** | 13.81^{Θ} | 0.0239** | 558.64 |
| (amorphous) | | | | | | |
| Lactose | — | 28 | | 28 | | |
| Microcrystalline | — | 46.95 | | 44.864 | | |
| Cellulose | | | | | | |
| Hydroxypropyl | — | 3 | | 3 | | |
| Celluose | | | | | | |
| Croscarmellose | — | 3 | | 3 | | |
| Sodium | | | | | | |
| Sodium Bicarbonate | — | 2 | 0.0238 | 0 | 0 | 84.01 |
| L-arginine | — | 0 | 0 | 4.1 | 0.0235 | 174.20 |
| Sodium Lauryl | — | 1 | | 1 | | |
| Sulphate | | | | | | |
| Magnesium Stearate | — | 2 | | 2 | | |
| Butylated Hydroxy | — | 0.12 | | 0.112 | | |
| Anisole | | | | | | |
| Butylated Hydroxy | — | 0.12 | | 0.112 | | |
| Toluene | | | | | | |
| Batch Size | | 400 g | | 400 g | | |

Human Biostudy

| | | | |
|---|---|---|---|
| AUC (nM*hr) | 163.5 | 155.8 | 152.2 |
| C-max (nM) | 34.33 | 36.8 | 34.92 |
| T-max (hr) | 1 | 1.2 | 1.5 |
| T-½ (hr) | 7.4 | 7.4 | 7.4 |

^{†}1:1 molar ratio of atorvastatin free acid:alkalizing additive
^{Θ}Based on MW of atorvastatin calcium salt without impurity

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and attributes of the invention, and all such variations are intended to be included within the scope of the following claims.

What is claimed is:

1. A pharmaceutical composition comprising atorvastatin and an alkalizing additive selected from the group consisting of L-arginine and sodium bicarbonate, wherein the atorvastatin is atorvastatin free acid or a pharmaceutically acceptable salt thereof, and wherein the molar ratio of the alkalizing additive to atorvastatin is from 1:1 to 6:1 on an atorvastatin free acid basis.

2. The pharmaceutical composition of claim 1, wherein said pharmaceutically acceptable salt is selected from the group consisting of a calcium salt, a sodium salt, and a magnesium salt.

3. The pharmaceutical composition of claim 2, comprising atorvastatin calcium salt.

4. The pharmaceutical composition of claim 3, wherein said atorvastatin calcium salt is crystalline.

5. The pharmaceutical composition of claim 3, wherein said atorvastatin calcium salt is amorphous.

6. The pharmaceutical composition of claim 1, wherein said alkalizing agent (a) is present in a molar ratio selected from 4:1, 3:1 and 2:1 with atorvastatin on an atorvastatin free acid basis, and (b) comprises less than 5% by weight of said composition.

7. The pharmaceutical composition of claim 1, wherein said alkalizing agent (a) is present in a 1:1 molar ratio with atorvastatin on an atorvastatin free acid basis, and (b) comprises less than 5% by weight of said composition.

8. The pharmaceutical composition of claim 7, wherein said alkalizing agent is L-arginine.

9. The pharmaceutical composition of claim 7, wherein said alkalizing agent is sodium bicarbonate.

10. The pharmaceutical composition of claim 1, wherein the composition is an oral dosage unit.

11. The pharmaceutical composition of claim 10, wherein the composition comprises a monolithic tablet or comprises one layer of a bilayer tablet.

12. The pharmaceutical composition of claim 11 in the form of a bi-layer tablet, comprised of (a) a first layer comprised of said composition containing atorvastatin for immediate release upon oral administration, and (b) a second layer comprised of a pharmaceutically active agent other than atorvastatin in a formulation that provides immediate or controlled release of the active agent upon oral administration.

13. The pharmaceutical composition of claim 12, wherein the active agent in the second layer is selected from the group consisting of a cholesterol absorption inhibitor, an HDL-raising agent, a CETP inhibitor and an anti-diabetic agent.

14. The pharmaceutical composition of claim 13, wherein the first layer is further comprised of a DP-antagonist and the second layer is comprised of niacin formulated for controlled release.

15. The pharmaceutical composition of claim 12, further comprising a coating on the exterior surface of the tablet.

16. The pharmaceutical composition of claim 11 in the form of a monolithic tablet further comprising at least one additional active agent other than atorvastatin.

17. The pharmaceutical composition of claim 1, further comprising one or more excipients selected from the group consisting of binders, diluents, disintegrants, surfactants, lubricants and combinations thereof.

18. The pharmaceutical composition of claim 17 comprising amorphous atorvastatin calcium salt, sodium bicarbonate, lactose (anhydrous), microcrystalline cellulose, hydroxypropyl cellulose, croscarmellose sodium, sodium lauryl sulfate and magnesium stearate.

19. The pharmaceutical composition of claim 18 comprising amorphous atorvastatin calcium salt, L-arginine, lactose (anhydrous), microcrystalline cellulose, hydroxypropyl cellulose, croscarmellose sodium, sodium lauryl sulfate and magnesium stearate.

20. The pharmaceutical composition of claim 17, wherein the composition is a bulk granulation.
